(19) United States
(12) Patent Application Publication
(10) Pub. No.: US 2006/0280687 A1
(43) Pub. Date: Dec. 14, 2006

(54) Compounds for tissue imaging and methods of use thereof

(76) Inventors: Zhen Cheng, Mountain View, CA (US); Sanjiv Sam Gambhir, Portola Valley, CA (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 11/378,988

(22) Filed: Mar. 17, 2006

Related U.S. Application Data
(60) Provisional application No. 60/663,465, filed on Mar. 18, 2005.

(51) Int. Cl. A61K 49/00; C07D 403/02; C07H 5/06

(52) U.S. Cl. 424/9.6; 536/18.7; 548/427; 536/55.3; 536/53

(57) ABSTRACT

Briefly described, embodiments of this disclosure include cyanine/glucose compounds, methods of use, methods of imaging tissue, methods of imaging precancerous tissue, cancer, and tumors, methods of treating precancerous tissue, cancer, and tumors, methods of diagnosing precancerous tissue, cancer and tumors, methods of monitoring the progress of precancerous tissue, cancer and tumors, and the like.

### CROSS-REFERENCE TO RELATED APPLICATION

This application claims priority to U.S. provisional application entitled, “Cyanine compounds for use in tissue imaging,” having Ser. No. 60/663,465, filed on Mar. 18, 2005, which is entirely incorporated herein by reference.

### BACKGROUND

Positron-emission tomography (PET) has become a generally accepted technology for pre-clinical and clinical non-invasive imaging of diseases, especially cancer. Tracers such as 2-deoxy-2-[¹⁸F]fluoro-D-glucose ([¹⁸F]FDG) and other radiopharmaceuticals that have the ability to target specific cellular and molecular processes have contributed to the rapid progress of PET technology.

Currently the majority (˜95%) of clinical PET scans use [¹⁸F]FDG for diagnosis and staging of cancer, as well as a tool for monitoring disease recurrence and drug therapeutic efficacy during therapy. The success of [¹⁸F]FDG-PET for metabolic imaging of disease has prompted development of other glucose analogs labeled with less costly and/or therapeutic isotopes such as ^{99m}Tc and ^{186/188}Re.

Although radionuclide imaging modalities display some advantages including intrinsically high sensitivity, capability of quantitation, and ability to image a human subject, they often suffer from relatively low spatial resolution, high cost, and significant radiation to personnel. Moreover, imaging probes with a short half-life are needed for PET imaging and for on-site cyclotron and radiochemistry laboratories. Furthermore, the recent emergence of optical imaging (bioluminescence and fluorescence imaging) as imaging modalities to study biological events in vitro and in vivo require new probes that do not involve radiation, and that are inexpensive yet highly sensitive. The acquisition times for optical imaging can be as short as seconds, allowing use of these techniques in high-throughput applications, if suitable probes are developed.

In recent years, the use of near-infrared fluorescent imaging as a diagnostic and detection tool has grown, in part because of the strong tissue penetration ability of light in the near-infrared (NIR) region (650-900 nm wavelengths). Near-infrared imaging is a viable method to non-invasively monitor disease states at a molecular level, to detect localized cancer areas, and to assess antitumor efficacy of therapeutic drugs. There remains a need, however, for compounds suitable for use in near-infrared optical imaging.

### SUMMARY

Briefly described, embodiments of this disclosure include cyanine/glucose compounds, methods of use, methods of imaging tissue, methods of imaging precancerous tissue, cancer, and tumors, methods of treating precancerous tissue, cancer, and tumors, methods of diagnosing precancerous tissue, cancer and tumors, methods of monitoring the progress of precancerous tissue, cancer and tumors, and the like.

An embodiment of a cyanine/glucose compound, among others, includes: a cyanine compound that fluoresces in the near infrared region attached to a glucose compound.

An embodiment of a method for imaging tissue, among others, includes: contacting a tissue with a cyanine/glucose compound.

An embodiment is provided of a fluorescent probe prepared according to a process, among others, that includes: reacting a cyanine compound having a moiety with a glucose compound having a moiety, wherein the moiety of the cyanine compound is capable of reacting with the moiety of the glucose compound to form a cyanine/glucose compound.

An embodiment is provided of a fluorescent probe prepared according to a process, among others, that includes: reacting a cyanine compound having a moiety capable of reacting with an amine with a glucose compound having an amine moiety.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1E show a general structure for cyanine compounds (FIG. 1A) and various, more specific structure for cyanine compounds (FIGS. 1B-1C) and a specific cyanine compound having an end functionalized alkyl (FIG. 1D).

FIG. 2 shows a synthetic reaction scheme for preparation of a compound by reacting a cyanine dye, Cy5.5, with D-glucosamine, to prepare a conjugate referred to herein as Cy5.5-2DG.

FIGS. 3A-3D show the UV absorption spectra of Cy5.5 dye (FIG. 3A) and of Cy5.5-2DG conjugate (FIG. 3B) and the fluorescence spectra of Cy5.5 dye (FIG. 3C) and of Cy5.5-2DG conjugate (FIG. 3D).

FIGS. 4A-4B are HPLC chromatograms of Cy5.5-2DG before (FIG. 4A) and after incubation in mouse serum for 30 minutes (FIG. 4B).

FIGS. 5A-5D are light images (FIGS. 5A-5B) and fluorescent images (FIGS. 5C-5D) of human glioblastoma cells U87MG incubated in vitro with Cy5.5-2DG for one hour at 37° C. (FIGS. 5A and 5C) and at 4° C. (FIGS. 5B and 5D).

FIGS. 6A-6D are light images (FIGS. 6A, 6B) and fluorescent images (FIGS. 6C and 6D) of C6 rat glioma cells incubated in vitro with Cy5.5-2DG for one hour at 37° C. (FIGS. 6A and 6C) and at 4° C. (FIGS. 6B and 6D).

FIGS. 7A-7D are light images (FIGS. 7A and 7B) and fluorescent images (FIGS. 7C and 7D) of human melanoma cells A375M incubated in vitro with Cy5.5-2DG for one hour at 37° C. (FIGS. 7A and 7C) and at 4° C. (FIGS. 7B and 7D).

FIGS. 8A-8D are light images (FIGS. 8A and 8B) and fluorescent images (FIGS. 8C and 8D) of murine melanoma cells B16F10 incubated in vitro with Cy5.5-2DG for one hour at 37° C. (FIGS. 8A and 8C) and at 4° C. (FIGS. 8B and 8D).

FIGS. 9A-9D are light images (FIGS. 9A and 9B) and fluorescent images (FIGS. 9C and 9D) of human breast carcinoma cells MDA-MB-435 incubated in vitro with Cy5.5-2DG for one hour at 37° C. (FIGS. 9A and 9C) and at 4° C. (FIGS. 9B and 9D).

FIGS. 10A-10M are images of sedated, live mice in the prone position bearing a human glioblastoma xenograft in the right foreleg as a function of time after administration of 500 pmol of Cy5.5-2DG.

FIGS. 11A-11B are plots of fluorescence intensity, in photons/sec/cm²/steradian, as a function of time post injection of 500 pmol of Cy5.5-2DG, in hours, in normal tissue (circles) and in tumor tissue (squares), where the fluorescence intensity is the average radiance (FIG. 11A) and the maximum radiance (FIG. 11B) for the mice shown in FIG. 10.

FIG. 11C is a plot of the ratio of fluorescence intensity of tumor tissue to the fluorescence intensity of healthy tissue, determined from the data presented in FIGS. 11A-11B, as a function of time post injection of 500 pmol of Cy5.5-2DG, in hours, where the ratio is determined for the measured average radiance (squares) and for the maximum radiance (circles).

FIGS. 12A-12M are images of sedated, live mice in the lateral position bearing a human glioblastoma xenograft in the right foreleg as a function of time after administration of 500 pmol of Cy5.5-2DG.

FIGS. 13A-13B are plots of fluorescence intensity, in photons/sec/cm²/steradian, as a function of time post injection of 500 pmol of Cy5.5-2DG, in hours, in normal tissue (circles) and in tumor tissue (squares), where the fluorescence intensity is the average radiance (FIG. 13A) and the maximum radiance (FIG. 13B) for the mice shown in FIG. 12.

FIG. 13C is a plot of the ratio of fluorescence intensity of tumor tissue to the fluorescence intensity of healthy tissue, determined from the data presented in FIGS. 13A-13B, as a function of time post injection of 500 pmol of Cy5.5-2DG, in hours, where the ratio is determined for the measured average radiance (squares) and for the maximum radiance (circles).

FIGS. 14A-14J are images of sedated, live mice in the prone position bearing a human glioblastoma xenograft in the right foreleg as a function of time after administration of 50 pmol of Cy5.5-2DG.

FIGS. 15A-15B are plots of fluorescence intensity, in photons/sec/cm²/steradian, as a function of time post injection of 50 pmol of Cy5.5-2DG, in hours, in normal tissue (circles) and in tumor tissue (squares), where the fluorescence intensity is the average radiance (FIG. 15A) and the maximum radiance (FIG. 15B) for the mice shown in FIG. 14.

FIG. 15C is a plot of the ratio of fluorescence intensity of tumor tissue to the fluorescence intensity of healthy tissue, determined from the data presented in FIGS. 15A-15B, as a function of time post injection of 50 pmol of Cy5.5-2DG, in hours, where the ratio is determined for the measured average radiance (squares) and for the maximum radiance (circles).

FIGS. 16A-16J are images of sedated, live mice in the lateral position bearing a human glioblastoma xenograft in the right foreleg as a function of time after administration of 50 pmol of Cy5.5-2DG.

FIGS. 17A-17B are plots of fluorescence intensity, in photons/sec/cm²/steradian, as a function of time post injection of 50 pmol of Cy5.5-2DG, in hours, in normal tissue (circles) and in tumor tissue (squares), where the fluorescence intensity is the average radiance (FIG. 17A) and the maximum radiance (FIG. 17B) for the mice shown in FIG. 16.

FIG. 17C is a plot of the ratio of fluorescence intensity of tumor tissue to the fluorescence intensity of healthy tissue, determined from the data presented in FIGS. 17A-17B, as a function of time post injection of 50 pmol of Cy5.5-2DG, in hours, where the ratio is determined for the measured average radiance (squares) and for the maximum radiance (circles).

FIGS. 18A-18L are images of sedated, live mice in the lateral position bearing a human melanoma xenograft in the right foreleg, as a function of time after administration of 500 pmol of Cy5.5-2DG, where three mice (n=3) were imaged at 2 hours post-injection (FIGS. 18A-18C), 3 hours post-injection (FIGS. 18D-18F), 24 hours post-injection (FIGS. 18G-18I), and 48 hours post-injection (FIGS. 18J-18L).

FIG. 19 is an image of organs removed from a mouse 45 hours post-injection of 500 pmol of Cy5.5-2DG.

FIGS. 20A-20F are photomicrographs taken at 10× magnification (FIGS. 20A-20C) and 60× magnification (FIGS. 20D-20F) of tumor tissue 24 hours post-injection of 50 pmol of Cy5.5-2DG, where FIGS. 20A and 20D are light images, FIGS. 20B and 20E are fluorescent images, and FIGS. 20C and 20F are overlay images.

FIGS. 21A-21F are photomicrographs taken at 10× magnification (FIGS. 21A-21C) and 60× magnification (FIGS. 21D-21F) of tumor tissue 62 hours post-injection of 50 pmol of Cy5.5-2DG, where FIGS. 21A and 21D are light images, FIGS. 21B and 21E are fluorescent images, and FIGS. 21C and 21F are overlay images.

FIGS. 22A-22F are photomicrographs of sedated, live tumor-bearing mice in the lateral position taken with a small animal imaging system from ART eXplore Optix (ART Inc./GE Healthcare) (FIGS. 22A-22C) and with the imaging system IVIS® (FIGS. 22D-22F) system 5 hours after administration of 500 pmol of Cy5.5-2DG.

FIGS. 23A-23F are photomicrographs of sedated, live tumor-bearing mice in the lateral position taken with a small animal imaging system from ART eXplore Optix (ART Inc./GE Healthcare) (FIGS. 23A-23C) and with the imaging system IVIS® (FIGS. 23D-23F) system 66 hours after administration of 500 pmol of Cy5.5-2DG.

### DETAILED DESCRIPTION

Embodiments of the present disclosure will employ, unless otherwise indicated, techniques of synthetic organic chemistry, biochemistry, molecular biology, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Definitions

In describing and claiming the disclosed subject matter, the following terminology will be used in accordance with the definitions set forth below.

By “administration” is meant introducing a compound into a subject. The preferred route of administration of the compounds is intravenous. However, any route of administration, such as oral, topical, subcutaneous, peritoneal, intraarterial, inhalation, vaginal, rectal, nasal, introduction into the cerebrospinal fluid, or instillation into body compartments can be used.

As used herein, the term “host”, “organism”, “individual” or “subject” includes humans, mammals (e.g., cats, dogs, horses, etc.), living cells, and other living organisms. A living organism can be as simple as, for example, a single eukaryotic cell or as complex as a mammal. “Patient” refers to an individual or subject who has undergone, is undergoing, or will undergo treatment.

“Cancer”, as used herein, shall be given its ordinary meaning, as a general term for diseases in which abnormal cells divide without control. Cancer cells can invade nearby tissues and can spread through the bloodstream and lymphatic system to other parts of the body.

There are several main types of cancer, for example, carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma is cancer that begins in the cells of the immune system.

When normal cells lose their ability to behave as a specified, controlled and coordinated unit, a tumor is formed. Generally, a solid tumor is an abnormal mass of tissue that usually does not contain cysts or liquid areas (some brain tumors do have cysts and central necrotic areas filled with liquid). A single tumor may even have different populations of cells within it, with differing processes that have gone awry. Solid tumors may be benign (not cancerous), or malignant (cancerous). Different types of solid tumors are named for the type of cells that form them. Examples of solid tumors are sarcomas, carcinomas, and lymphomas. Leukemias (cancers of the blood) generally do not form solid tumors.

Representative cancers include, but are not limited to, bladder cancer, breast cancer, colorectal cancer, endometrial cancer, head & neck cancer, leukemia, lung cancer, lymphoma, melanoma, non-small-cell lung cancer, ovarian cancer, prostate cancer, testicular cancer, uterine cancer, cervical cancer, thyroid cancer, gastric cancer, brain stem glioma, cerebellar astrocytoma, cerebral astrocytoma, glioblastoma, ependymoma, Ewing's sarcoma family of tumors, germ cell tumor, extracranial cancer, Hodgkin's disease, leukemia, acute lymphoblastic leukemia, acute myeloid leukemia, liver cancer, medulloblastoma, neuroblastoma, brain tumors generally, non-Hodgkin's lymphoma, osteosarcoma, malignant fibrous histiocytoma of bone, retinoblastoma, rhabdomyosarcoma, soft tissue sarcomas generally, supratentorial primitive neuroectodermal and pineal tumors, visual pathway and hypothalamic glioma, Wilns'tumor, acute lymphocytic leukemia, adult acute myeloid leukemia, adult non-Hodgkin's lymphoma, chronic lymphocytic leukemia, chronic myeloid leukemia, esophageal cancer, hairy cell leukemia, kidney cancer, multiple myeloma, oral cancer, pancreatic cancer, primary central nervous system lymphoma, skin cancer, small-cell lung cancer, among others.

A tumor can be classified as malignant or benign. In both cases, there is an abnormal aggregation and proliferation of cells. In the case of a malignant tumor, these cells behave more aggressively, acquiring properties of increased invasiveness. Ultimately, the tumor cells may even gain the ability to break away from the microscopic environment in which they originated, spread to another area of the body (with a very different environment, not normally conducive to their growth), and continue their rapid growth and division in this new location. This is called metastasis. Once malignant cells have metastasized, achieving a cure is more difficult.

Benign tumors have less of a tendency to invade and are less likely to metastasize. Brain tumors spread extensively within the brain but do not usually metastasize outside the brain. Gliomas are very invasive inside the brain, even crossing hemispheres. They do divide in an uncontrolled manner, though. Depending on their location, they can be just as life threatening as malignant lesions. An example of this would be a benign tumor in the brain, which can grow and occupy space within the skull, leading to increased pressure on the brain.

It should be noted that precancerous cells, cancer, tumors are often used interchangeably in the disclosure.

A “linking moiety” or “linking group”, as used herein, is a group that connects the cyanine compound with a glucose compound. The linking moiety can include, but is not limited to, an amid linker, an ester linker, a hydrazone linker, a sulfide linker,and the like.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. For example the term “alkyl” can refer to straight or branched chain hydrocarbon groups, such as methyl, ethyl, n-propyl, i-propyl, n-butyl, i-butyl, t-butyl, pentyl, hexyl, heptyl, octyl, and the like. Likewise, cycloalkyls have from about 3 to about 10 carbon atoms in their ring structure, and alternatively about 5, 6 or 7 carbons in the ring structure. The term “alkyl” is also defined to include halosubstituted alkyls.

Moreover, the term “alkyl” (or “lower alkyl”) includes “substituted alkyls”, which refers to alkyl moieties having substituents replacing a hydrogen on one or more carbons of the hydrocarbon backbone. Such substituents may include, for example, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain may themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CN and the like. Cycloalkyls may be further substituted with alkyls, alkenyls, alkoxys, alkylthios, aminoalkyls, carbonyl-substituted alkyls, —CN, and the like.

The term “aryl” refers to 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.”

The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like.

The term “aryl” also includes polycyclic ring systems having two or more cyclic rings in which two or more carbons are common to two adjoining rings (the rings are “fused rings”) wherein at least one of the rings is aromatic (e.g., the other cyclic rings may be cycloalkyls, cycloalkenyls, cycloalkynyls, aryls and/or heterocyclyls).

The terms “halogen” and “halo” refer to fluorine, chlorine, bromine, and iodine.

The terms “heterocycle”, “heterocyclic”, “heterocyclic group” or “heterocyclo” refer to fully saturated or partially or completely unsaturated, including aromatic (“heteroaryl”) or nonaromatic cyclic groups (for example, 3 to 13 member monocyclic, 7 to 17 member bicyclic, or 10 to 20 member tricyclic ring systems), which have at least one heteroatom in at least one carbon atom-containing ring. Each ring of the heterocyclic group containing a heteroatom may have 1, 2, 3, or 4 heteroatoms selected from nitrogen atoms, oxygen atoms, and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system. The rings of multi-ring heterocycles may be either fused, bridged, and/or joined through one or more spiro unions.

The terms “substituted heterocycle”, “substituted heterocyclic”, “substituted heterocyclic group” and “substituted heterocyclo” refer to heterocycle, heterocyclic, and heterocyclo groups substituted with one or more groups preferably selected from alkyl, substituted alkyl, alkenyl, oxo, aryl, substituted aryl, heterocyclo, substituted heterocyclo, carbocyclo (optionally substituted), halo, hydroxy, alkoxy (optionally substituted), aryloxy (optionally substituted), alkanoyl (optionally substituted), aroyl (optionally substituted), alkylester (optionally substituted), arylester (optionally substituted), cyano, nitro, amido, amino, substituted amino, lactam, urea, urethane, sulfonyl, and the like, where optionally one or more pair of substituents together with the atoms to which they are bonded form a 3 to 7 member ring.

General Discussion

Briefly described, embodiments of this disclosure, among others, include cyanine/glucose compounds, methods of use, methods of imaging tissue, methods of imaging precancerous tissue, cancer, and tumors, methods of treating precancerous tissue, cancer, and tumors, methods of diagnosing precancerous tissue, cancer and tumors, methods of monitoring the progress of precancerous tissue, cancer and tumors, and the like. Embodiments of the cyanine/glucose compounds can be used to image tissue (e.g., cancer, precancerous tissue, and turmors) becase the cyanine/glucose compounds fluoresce (e.g., at about 500 to 900 nm), and can be detected by an appropriate detection system (e.g., fluorescence imaging systems and the like). In addition, the cyanine/glucose compounds can be used to detect (and visualize) tissue or particular types of tissue in an organ or larger tissue mass in vitro as well as in vivo, which decreases time and expense relative to other systems. It should be noted that embodiments of the present disclosure do not involve any ionizing radiation, are inexpensive, and are highly sensitive. In addition, embodiments of the present disclosure allow for high throughput screening since the acquisition times for obtaining a good image using optical imaging can be as short as a few seconds.

Cyanine/Glucose Compounds

The cyanine/glucose compound includes a cyanine compound attached (e.g., bonded directly or indirectly via a linking moiety) to a glucose compound. Although not intending to be bound by theory, cyanine compounds are characterized by a resonance structure including a polymethine chain bearing two terminal nitrogen atoms and an overall positive structure. “Cyanine compounds” generically refer to a compound having two substituted or unsubstituted nitrogen-containing heterocyclic rings joined by an unsaturated bridge. An exemplary general structure of a cyanine compound is: 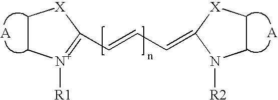
where X is selected from, but is not limited to, O, NR, C(CH₃)₂, or S; R, R1, and R2 are independently selected from, but are not limited to, alkyl groups, aryl groups, substituted alkyl groups, and substituted aryl groups; A is a cyclic multi-ring or a fused multi-ring; and n is 1, 2, or 3. Exemplary cyanine compounds having this general form are described for example in U.S. Pat. Nos. 6,995,274, 6,683,188, 6,649,335, 6,437,141, 6,224,644, 6,114,350, 6,197,956, 6,027,709, and 5,268,486, which are each incorporated by reference herein. Such compounds are commercially available from, for example, Amersham Biosciences, as compounds referred to as CyDye®.

The cyanine compound fluoresces in the near infrared region (e.g., about 500-900 nm). Modification of the heterocycle and/or polymethine chain results in dyes having a range of excitation and emission wavelengths. In various embodiments, the cyanine dye is one having an excitation (absorbance) wavelength of about 550-900 nm, about 550-750 nm, and about 600-700, and an emission wavelength of about 570-900 nm, about 570-780 nm and about 620-720 nm. In a specific embodiment, the cyanine compound has an absorbance wavelength of about 675 nm and a fluorescence emission wavelength of about 694 nm.

In an embodiment, a cyanine compound is reacted with a glucose compound (e.g., glucose or a derivative thereof). As used herein, “glucose derivative” includes glucose modified by reaction at one or more of its hydroxyl moieties. For example, reaction of glucose with a cyanine dye results in the cyanine dye attached to a glucose derivative. Reaction of glucose with a compound bearing a functional group to provide glucose functionalized for reaction with specific moieties is also an example of a glucose derivative.

The glucose compound can include, but is not limited to, glucose and derivatives thereof, D-glucose (dextraglucose) and derivatives thereof, glucosamine (2-amino-2-deoxyglucose) and derivatives thereof, glucose-6-phosphate and derivatives thereof, glucose-1-phosphate and derivatives thereof, and deoxyglucose (e.g., 2-deoxyglucose) and derivatives thereof

For example, an exemplary fluorescent probe was prepared from a cyanine compound and a glucose derivative, glucosamine, according to the procedure described in Example 1. D-glucosamine and a cyanine compound bearing a single N-hydroxysuccinime (NHS) moiety, Cy5.5®-NHS, were mixed together and incubated overnight. The reaction scheme is shown in FIG. 2 and resulted in a cyanine dye-glucose derivative compound referred to herein as “Cy5.5-2DG”, the nomenclature refers to the fact that the Cy5.5 dye is attached to carbon number 2 of D-glucosamine. Thus, in a preferred embodiment, a compound including a cyanine dye and a derivative of glucose is prepared by reacting the cyanine dye with glucose or a glucose derivative to obtain a compound where the cyanine dye is attached to the 2 carbon in glucose. Additional details are provided in the Examples.

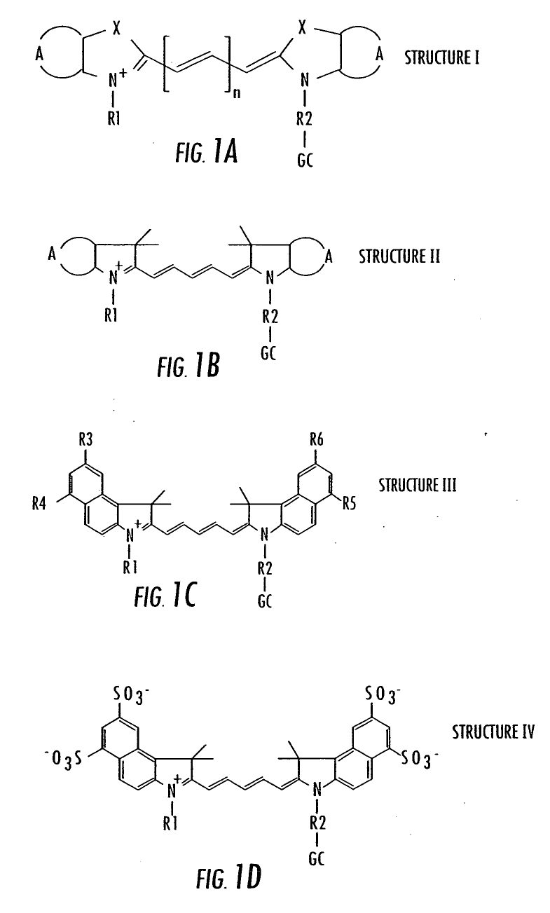

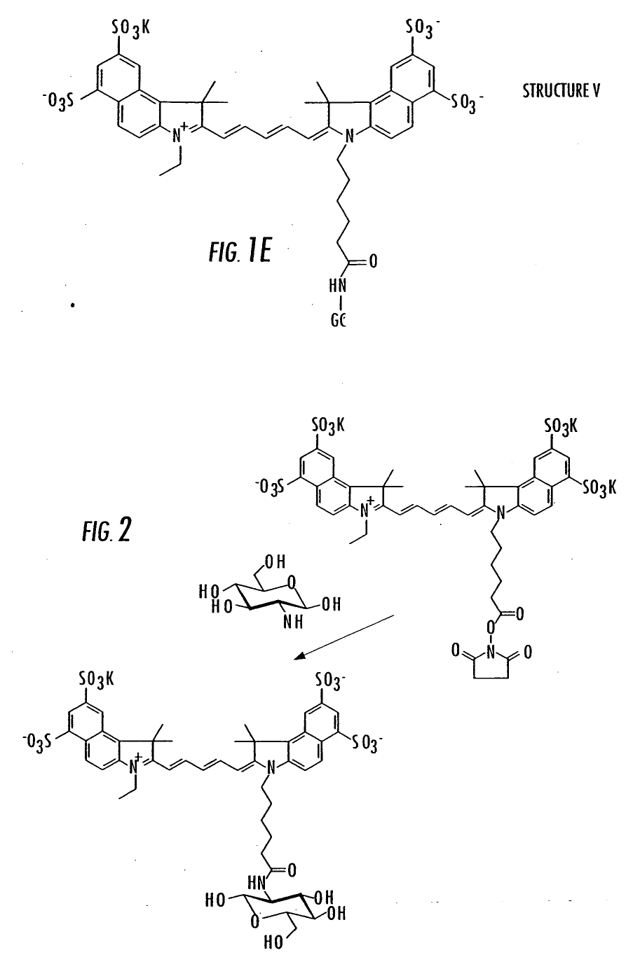

As mentioned above, the cyanine/glucose compound includes, but is not limited to, a compound including a cyanine compound (e.g., that fluoresces in the near infrared region) attached (e.g., bonded directly or indirectly using a linking moiety) to a glucose compound. Exemplary embodiments of the cyanine/glucose compound are shown in FIGS. 1A through 1E. In an embodiment, the cyanine/glucose compound includes, but is not limited to, a compound including a cyanine compound (e.g., that fluoresces in the near infrared region) attached (e.g., bonded directly or indirectly using a linking moiety) to two or more glucose compounds (e.g., two different positions on the cyanine compound and/or two different positions on a single linking moiety). For structures I-V, X is selected from, but is not limited to, one of the following: O, NR, C(CH₃)₂, and S. R, R1, and R2 are each independently selected from, but are not limited to, one of the following: an alky group, an aryl group, a substituted alkyl, and a substituted aryl. A is selected from, but is not limited to, a cyclic multi-ring and a fused multi-ring. Subscript n is 1, 2, or 3. GC is a glucose compound, which is described in more detail above. It should also be noted that a linking moiety can be disposed between R2 and GC. Linking moieties can include, but are not limited to, an amide linkage, a hydrazone linker, a sulfide linker, and the like. In an embodiment, the “K” group (potassium) on structure V can be removed.

For structure III, R3, R4, R5, and R6 are each independently selected from, but are not limited to, a sulfonic acid group and a sulfonate group.

Methods of Use

As mentioned above, embodiments of this disclosure include, but are not limited to: methods of imaging tissue; methods of imaging precancerous tissue, cancer, and tumors; methods of treating precancerous tissue, cancer, and tumors; methods of diagnosing precancerous tissue, cancer, and tumors; methods of monitoring the progress of precancerous tissue, cancer, and tumors; methods of imaging abnormal tissue, and the like.

In general, the cyanine/glucose compound can be used in imaging cells or tissue. For example, the cyanine/glucose compound is provided to a host in an amount effective to result in uptake of the compound into the cells or tissue of interest. The host is then exposed to an appropriate radiation source (e.g., a light source) after a certain amount of time. In particular, the light source irradiates the subject or a particular area of the subject with light suitable to excite the cyanine/glucose compound. The cells or tissue that take up the cyanine/glucose compound can be detected using an appropriate detection system (e.g., fluorescence systems).

In an embodiment, the cyanine/glucose compound can be used in imaging cancerous cells, precancerous cells, and tumors. It should be noted that the cyanine/glucose compounds are preferentially taken up by cancerous cells, precancerous cells, and tumors. Thus, the cyanine/glucose compounds find use both in diagnosing cancer and in treating cancer.

In diagnosing the presence of cancerous cells, precancerous cells, and tumors in a subject, a cyanine/glucose compound is administered to the subject in an amount effective to result in uptake of the cyanine/glucose compound into the cells. After administration of the compound, cells that take up the cyanine/glucose compound are detected by, for example, optical imaging of the patient. Typically, the detection is done within about 24-48 hours of administering the cyanine/glucose compound by irradiating the subject or an area of the subject suspected of having cancer with light suitable to excite the cyanine/glucose compound. Embodiments of the present disclosure can non-invasively image tissue at a depth of about 0 cm to 2.2 cm in an animal or patient. Since cancer cells take up the compound at a rate that is about 1.5-2 times greater than healthy tissue, and since the cyanine/glucose compound remains in the cancer cells for as long as 100 hours after administration of the compound, cancer cells, pre-cancer cells, or tumors can be visualized.

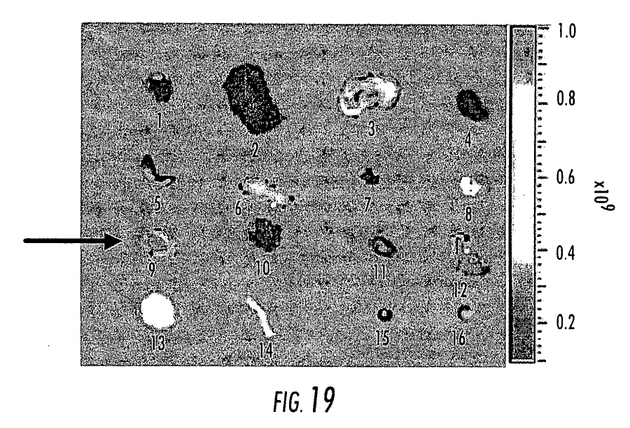

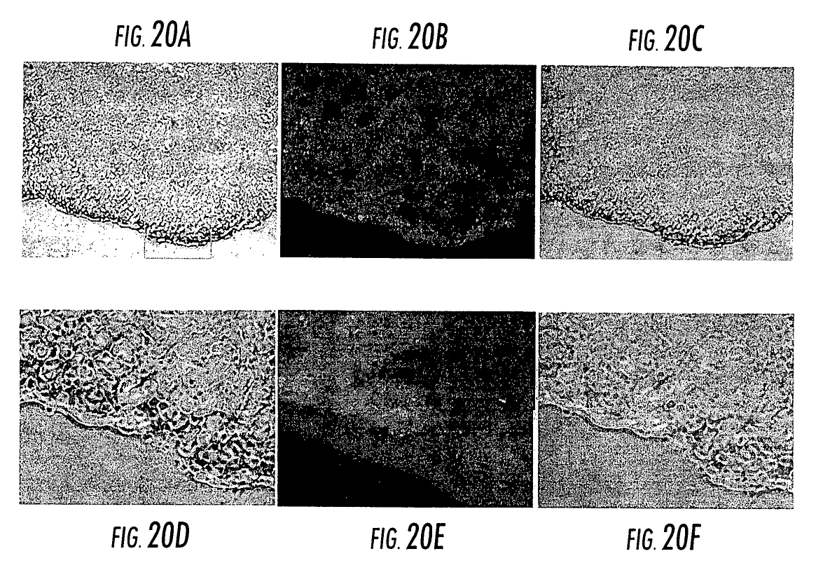

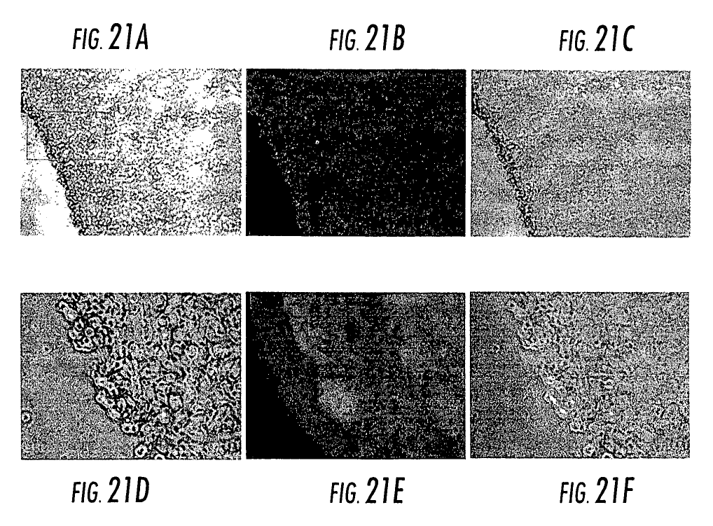

In another embodiment, the cyanine/glucose compounds can be used in treating cancer that has been previously diagnosed by a method described herein or by another method. The cyanine/glucose compounds finds use in both surgical treatment and in chemical treatment of cancerous tissue. In patients where cancerous tissue is to be surgically removed, the cyanine/glucose compound is administered prior to and/or coincident with the surgical procedure. The cancerous tissue is illuminated with a light source having a wavelength in the excitation spectrum of the compound. An attending medical provider can then, with suitable equipment, directly visualize the illuminated tissue for fluorescence emanating from the site. As described in detail in the Examples and as illustrated in FIGS. 20-21, the boundaries of the cancer tissue are readily apparent, easing surgical resection of the tissue and assuring a more complete removal of the cancer with less removal of healthy adjacent tissue.

The cyanine/glucose compounds also find use in patients undergoing chemotherapy, to aid in visualizing the response of tumor tissue to the treatment. In this embodiment, the cancer tissue is typically visualized and sized prior to treatment, and periodically during chemotherapy to monitor the tumor size.

The cyanine/glucose compounds also find use as a screening tool in vitro to select compounds for use in treating cancer. The size of an in vitro tumor can be easily monitored in the presence of candidate drugs by incubating the cells with the cyanine/glucose compounds during or after incubation with one or more candidate drugs.

It should be noted that the amount effective to result in uptake of the compound into the cells or tissue of interest will depend upon a variety of factors, including for example, the activity of the specific composition employed; the specific composition employed; the age, body weight, general health, sex, and diet of the host; the time of administration; the route of administration; the rate of excretion of the specific compound employed; the duration of the treatment; the existence of other drugs used in combination or coincidental with the specific composition employed; and like factors well known in the medical arts.

Dosage Forms

Unit dosage forms of the compounds of this disclosure may be suitable for oral, mucosal (e.g., nasal, sublingual, vaginal, buccal, or rectal), parenteral (e.g., intramuscular, subcutaneous, intravenous, intra-arterial, or bolus injection), topical, or transdermal administration to a patient. Examples of dosage forms include, but are not limited to: tablets; caplets; capsules, such as hard gelatin capsules and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the compositions of the disclosure typically vary depending on their use. For example, a parenteral dosage form may contain smaller amounts of the active ingredient than an oral dosage form used to treat the same condition or disorder. These and other ways in which specific dosage forms encompassed by this disclosure vary from one another will be readily apparent to those skilled in the art (See, e.g., Remington's Pharmaceutical Sciences, 18th ed., Mack Publishing, Easton, Pa. (1990)).

Typical compositions and dosage forms of the compositions of the disclosure can include one or more excipients. Suitable excipients are well known to those skilled in the art of pharmacy or pharmaceutics, and non-limiting examples of suitable excipients are provided herein. Whether a particular excipient is suitable for incorporation into a composition or dosage form depends on a variety of factors well known in the art including, but not limited to, the way in which the dosage form will be administered to a patient. For example, oral dosage forms, such as tablets or capsules, may contain excipients not suited for use in parenteral dosage forms. The suitability of a particular excipient may also depend on the specific active ingredients in the dosage form. For example, the decomposition of some active ingredients can be accelerated by some excipients, such as lactose, or by exposure to water. Active ingredients that include primary or secondary amines are particularly susceptible to such accelerated decomposition.

The disclosure encompasses compositions and dosage forms of the compositions of the disclosure that can include one or more compounds that reduce the rate by which an active ingredient will decompose. Such compounds, which are referred to herein as “stabilizers,” include, but are not limited to, antioxidants such as ascorbic acid, pH buffers, or salt buffers. In addition, pharmaceutical compositions or dosage forms of the disclosure may contain one or more solubility modulators, such as sodium chloride, sodium sulfate, sodium or potassium phosphate, or organic acids. An exemplary solubility modulator is tartaric acid.

Like the amounts and types of excipients, the amounts and specific type of active ingredient in a dosage form may differ depending on various factors. It will be understood, however, that the total daily usage of the compositions of the present disclosure will be decided by the attending physician or other attending professional within the scope of sound medical judgment. The specific effective dose level for any particular host will depend upon a variety of factors, including for example, the activity of the specific composition employed; the specific composition employed; the age, body weight, general health, sex, and diet of the host; the time of administration; the route of administration; the rate of excretion of the specific compound employed; the duration of the treatment; the existence of other drugs used in combination or coincidental with the specific composition employed; and like factors well known in the medical arts. For example, it is well within the skill of the art to start doses of the composition at levels lower than those required to achieve the desired effect and to gradually increase the dosage until the desired effect is achieved.

Kits

This disclosure encompasses kits, which may include, but are not limited to, a cyanine/glucose compound and directions (written instructions for their use). The components listed above can be tailored to the particular cancer or tumor to be monitored. The kit can further include appropriate reagents known in the art for administering various combinations of the components listed above to the host organism or patient.

### EXAMPLES

Now having described the embodiments of the cyanine/glucose compounds and methods of use, in general, Examples 1 through 3 describe some additional embodiments of the cyanine/glucose compounds and methods of use. While embodiments of cyanine/glucose compounds and methods of use are described in connection with Examples 1 through 3 and the corresponding text and figures, there is no intent to limit embodiments of the cyanine/glucose compounds and methods of use to these descriptions. On the contrary, the intent is to cover all alternatives, modifications, and equivalents included within the spirit and scope of embodiments of the present disclosure.

Materials: Cy5.5 monofunctional NHS ester (Cy5.5-NHS) was purchased from Amersham Biosciences (Piscataway, N.J.). All other reagents were purchased from Sigma-Aldrich Chemical Co. (St. Louis, Mo.).

Methods: Matrix Assisted Laser Desorption/Ionization Time of Flight Mass Spectrometry (MALDI-TOF-MS) was performed by Stanford Protein and Nucleic Acid Biotechnology Facility, Stanford University.

HPLC was performed on a Dionex Summit® HPLC system (Dionex Corporation, Sunnyvale, Calif.) equipped with a 170U 4-Channel UV-Vis absorbance detector. UV detection wavelengths are 218 nm, 280 nm and 590 nm for all the experiments. Both semi-preparative (Zorbax SB-C18, 9.4 mm×250 mm) and analytical (Dionex Acclaim® 120 C18, 4.6 mm×250 mm) RP-HPLC columns were used. The mobile phase was solvent A, 0.1% trifluoroacetic acid (TFA) in water and solvent B, 0.1%TFA in acetonitrile (CH₃CN).

### Example 1

Synthesis of Cyanine-D-glucosamine Conjugate

D-glucosamine (30.2 mg) was dissolved in 302 μL H₂O to make a concentration of 463.7 mM solution. D-Glucosamine (34.5 μL, 16.0 μmol), sodium phosphate buffer (Na₂HPO₄, pH=9.0, 0.1 M, 805.5 μL) and Cy5.5-NHS (1.81 mg, 1.60 μmol) dissolved in 160 μL H₂O were then mixed together. After incubation at 4° C. for overnight in the dark, the reaction was quenched by adding 100 μL of 1% trifluoroacetic acid (TFA). The reaction scheme of the Cy5.5-2DG compound is shown in FIG. 2.

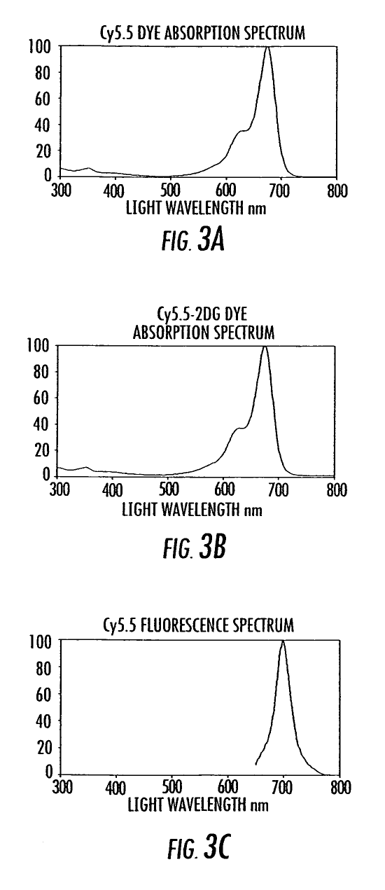

The crude product was then injected onto a semi-preparative HPLC column, where the flow rate was 3 mL/min, with the mobile phase starting from 5% solvent B (0.1%TFA in CH₃CN) and 95% solvent A (0.1%TFA in water) (0-3 min) to 65% solvent B and 35% solvent A at 33 min, then going to 85% solvent B and 15% solvent A (33-36 min), maintaining this solvent composition for another three minutes (36-39 min), and returning to initial solvent composition by 42 min. Product elution was monitored at both 218 and 590 nm using a UV-Vis detector. The product peak was collected, lyophilized, and identified by MALDI-TOF-MS. The chemical purity of product was determined by analytical HPLC (same gradient as used for semi-preparative HPLC; flow rate: 1.0 ml/min). The product was re-dissolved in saline at a concentration of 1 mg/mL, and stored in the dark at −80° C. until use. The absorbance spectrum of Cy5.5-2DG was rerecorded on an Agilent 8453 UV-visible ChemStation (Agilent Technologies, Wilmington, DE), and fluorescence spectrum of Cy5.5-2DG was measured on a Fluoromax-3 fluorophotometer (JOBIN YVON/HORIBA, Edison, N.J.). The UV/fluorescence spectra of Cy5.5 and of Cy5.5-2DG are shown in FIGS. 3A-3D.

### Example 2

In Vitro Characterization of Cyanine-D-glucosamine Conjugate

Human glioblastoma U87MG cells were cultured in Dulbecco's modified Eagle medium (DMEM, Invitrogen Life Technologies, Carlsbad, Calif.), high glucose plus 10% fetal bovine serum (FBS), and 1% penicillin-streptomycin. The cells were maintained in a humidified atmosphere of 5% CO₂ at 37° C., with the medium changed every other day. A confluent monolayer was detached with trypsin and dissociated into a single cell suspension for further cell culture.

For fluorescence microscopic studies, 1 M cells were cultured on 35 mm MatTek glass bottom culture dishes (Cat #: P35G-0-14-C, Ashland, Mas.). After 24 hours, the cells were washed with phosphate-buffered saline (PBS) and then incubated at 37° C. or 4° C. in the presence of 100 nM Cy5.5-2DG for 1 hour. Afterwards, cells were washed in 1 mL ice-cold PBS. The fluorescence signal of the cells was recorded using an Axiovert 200M fluorescence microscope (Carl Zeiss MicroImaging, Inc., Thornwood, N.Y.) equipped with a Cy5.5 filter set (Exciter, HQ 650/20 nm; Emitter, HQ 675/35 nm). An AttoArc HBO 100 W microscopic illuminator was used as a light source for fluorescence excitation. Images were taken using a thermoelectrically cooled charge-coupled device (CCD) (Micromax, model RTE/CCD-576, Princeton Instruments Inc., Trenton, N.J.) and analyzed using WinView Software version 1.6.2 (Princeton Instruments Inc., Trenton, N.J.)

### Example 3

In Vivo Characterization of Cyanine-D-glucosamine Conjugate

All animal studies were carried out in compliance with federal and local institutional rules for the conduct of animal experimentation. Female athymic nude mice (nu/nu, 10-12 weeks old), obtained from Charles River Laboratories (Boston, Mas.), were inoculated subcutaneously in the right foreleg with 5×10⁶ U87MG cultured glioblastoma cells suspended in 100 μL of PBS. Two to three weeks after inoculation, when the tumors reached 0.4-0.6 cm in diameter, the tumor bearing mice were subject to in vivo imaging studies.

In vivo fluorescence imaging was performed with a cooled charged-coupled device (CCD) camera (Xenogen IVIS™ 200 small animal imaging system, Xenogen, Alameda, Calif.). A Cy5.5 filter set was used for acquiring Cy5.5-2DG fluorescence in vivo. Identical illumination settings (lamp voltage, filters, f/stop, field of views, binning) were used for acquiring all images, and fluorescence emission was normalized to photons per second per centimeter squared per steradian (p/s/cm²/sr). Images were acquired and analyzed using Living Image® D2.5 software (Xenogen).

Mice were injected via tail vein with 50 or 500 pmol Cy5.5-2DG and subjected to optical imaging at various time points post injection with a sampling of multiple angles while remaining sedated. A mouse injected with 50 pmol of Cy5.5-2DG was euthanized at 45 h postinjection (p.i.), the tumor and major tissue and organs were dissected, and fluorescence image was obtained.

All the data are given as means ± SD of n independent measurements. Statistical analysis was performed using a Student's t-test. Statistical significance was assigned for P values <0.05. For determining tumor contrast, mean fluorescence intensities of the tumor area at the right shoulder of the animal (T) and of the corresponding area at the left shoulder (N) were calculated by means of the region of interest function of Living Image software(Xenogen) integrated with Igor (Wavemetrics, Lake Oswego, Oreg.). Dividing T by N yielded the contrast between tumor tissue and normal tissue.

### DISCUSSION

### Examples 1 through 3

An exemplary fluorescent probe was prepared from a cyanine compound and a glucose derivative, glucosamine, according to the procedure described in Example 1. D-glucosamine and a cyanine compound bearing a single N-hydroxysuccinime (NHS) moiety, Cy5.5®-NHS, were mixed together and incubated overnight. The reaction scheme is shown in FIG. 2 and resulted in a cyanine dye-glucose derivative compound referred to herein as “Cy5.5-2DG”, the nomenclature referring to the fact that the Cy5.5 dye is attached to carbon number 2 of D-glucosamine. Thus, in a preferred embodiment, a compound comprised of a cyanine dye and a derivative of glucose is prepared by reacting the cyanine dye with glucose or a glucose derivative to obtain a compound where the cyanine dye is attached to the 2 carbon in glucose.

The UV absorption spectra of Cy5.5® dye and of Cy5.5-2DG conjugate are shown in FIGS. 3A-3B, respectively. The absorption spectra of the neat Cy5.5® dye and of the Cy5.5-2DG probe are essentially identical with both the neat dye and the probe absorbing at about 675 nm, indicating that attachment of a glucose derivative to the dye did not measurably alter is absorption properties. The fluorescence spectra of Cy5.5® dye and of Cy5.5-2DG conjugate are shown in FIGS. 3C-3D, respectively. The emission spectra of the neat dye and of the probe are essentially the same, with both compounds exhibiting fluorescent emission at about 694 nm, indicating that attachment of a glucose derivative to the dye did not measurably alter is emission properties.

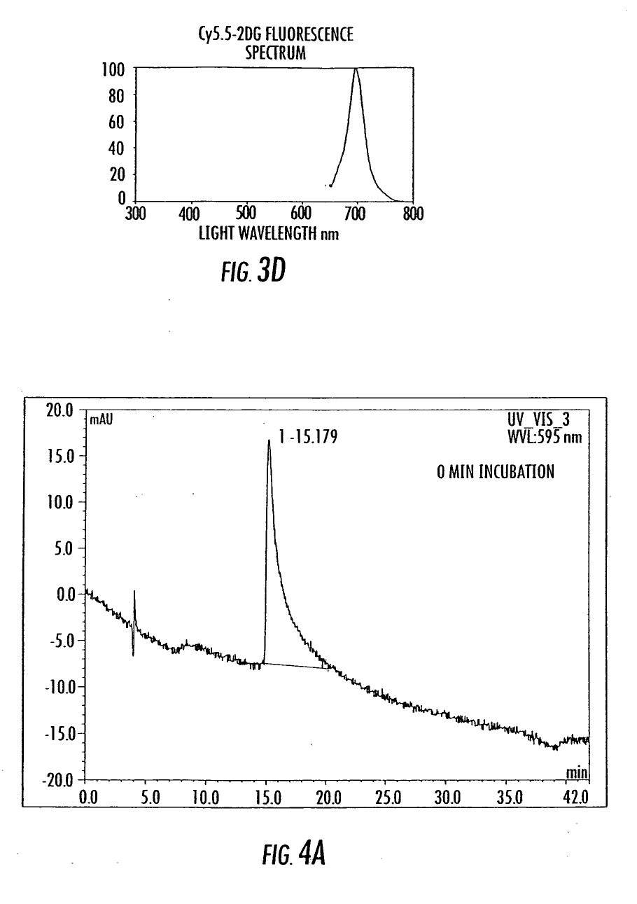

The stability of the Cy5.5-2DG compound was evaluated by incubating the compound in vitro in mouse serum and analyzing the serum as a function of time by high pressure liquid chromatography (HPLC). FIG. 4A shows an HPLC analysis of the Cy5.5-2DG compound in solution, after preparation according to the procedure in Example 1. The main peak is observed at 15.2 minutes. FIG. 4B shows the HPLC chromatogram taken after 30 minutes of incubation in mouse serum. The same peak (15.2 minutes) is still observed as the major peak, suggesting Cy5.5-2DG is stable over 30 minutes incubation with mouse serum.

Tumor uptake of the Cy5.5-2DG compound into human glioblastoma cells (U87MG) was determined by incubating the cells in vitro with the compound for one hour at either 37° C. or at 4° C., as described in Example 2. After incubation, the cells were washed with saline and the fluorescence signal of the cells was recorded using a fluorescence microscope equipped with a Cy5.5 filter set. The light images are shown in FIGS. 5A-5B and the fluorescent images are shown in FIGS. 5C-5D. In both of the light images (FIGS. 5A and 5B) tumor cells are visible in the field. However, the fluorescent images show that incubation at 37° C. resulted in considerably more uptake of the fluorescent compound by the cells, as evidenced by the fluorescing cell in the image of FIGS. 5C and the lack of fluorescing cells in the image of FIG. 5D.

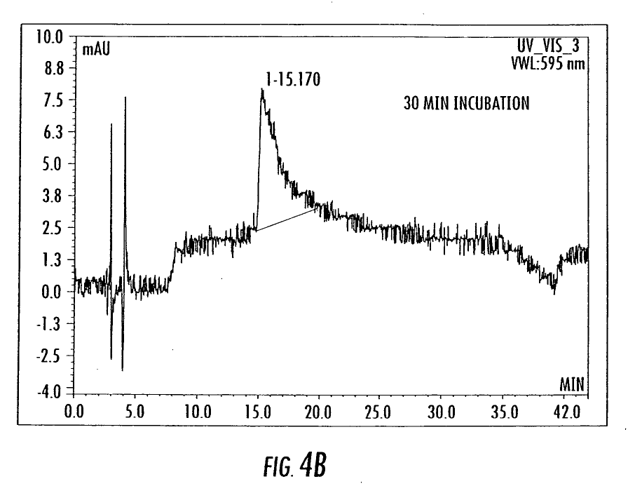

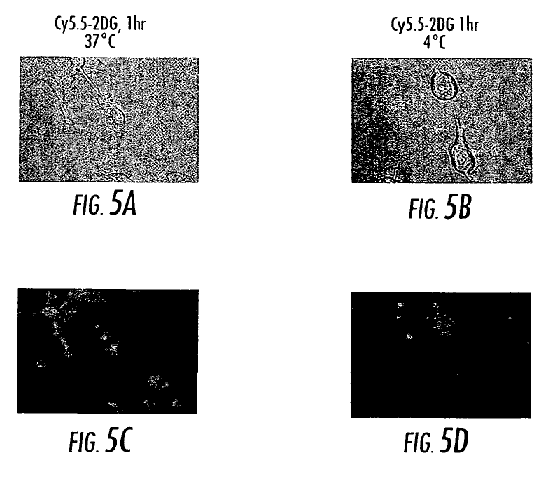

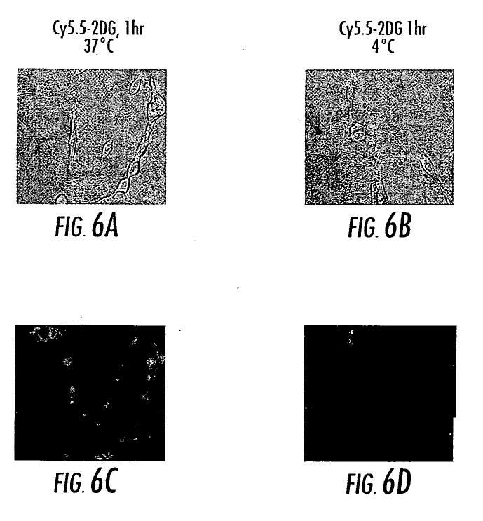

Additional in vitro studies were conducted with several other tumor cells lines, including C6 rat glioma cells. Following the procedure set forth in Example 2, the glioma cells were incubated for one hour at 37° C. or at 4° C., and light images and fluorescent images were taken. The light images are shown in FIGS. 6A and 6B and the fluorescent images in FIGS. 6C and 6D. Both light images show cells in the image field. The uptake of the dye into the cells when incubated at 37° C. is apparent from the distinct fluorescing cells visible in the fluorescent image of FIG. 6C. In contrast, cells incubated at 4° C. took up little, if any, of the compound, as evidenced by the lack of distinct fluorescing cells in FIG. 6D.

The results of in vitro incubation with A375M human melanoma cells and the Cy5.5-2DG compound are shown in FIGS. 7A-7D. The light images (FIGS. 7A and 7B) show cells in the optical field. The fluorescent images (FIGS. 7C and 7D) shows that the cells incubated at 37° C. (FIG. 7C) took in the compound, whereas cells incubated with the compound at 4° C. (FIG. 7D) had no apparent fluorescence.

FIGS. 8A and 8C show a light image (FIG. 8A) and a fluorescent image (FIG. 8C) of murine melanoma cells B16F10 incubated in vitro with Cy5.5-2DG for one hour at 37° C. Uptake of the compound into the cells is apparent from the distinct fluorescing cells visible in the fluorescent image of FIG. 8C. FIGS. 8B and 8D show a light image (FIG. 8B) and a fluorescent image (FIG. 8D) of the same type of cells incubated in vitro with Cy5.5-2DG for one hour at 4° C. Little, if any, of the fluorescent probe is take up by the cells at this lower temperature.

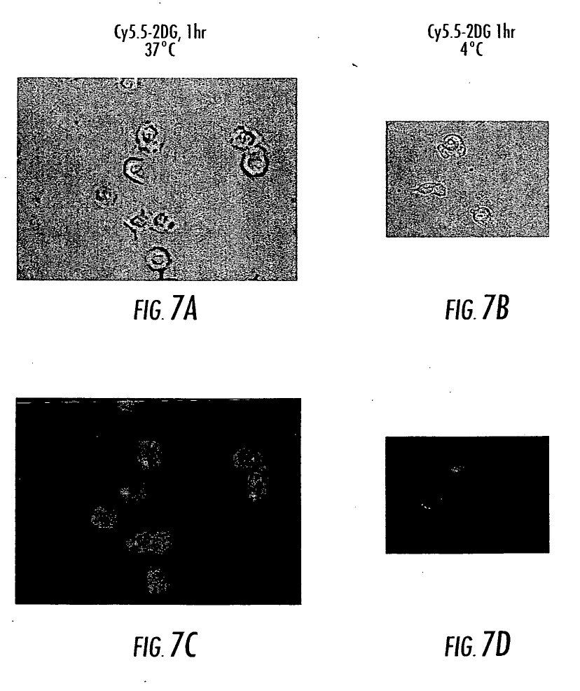

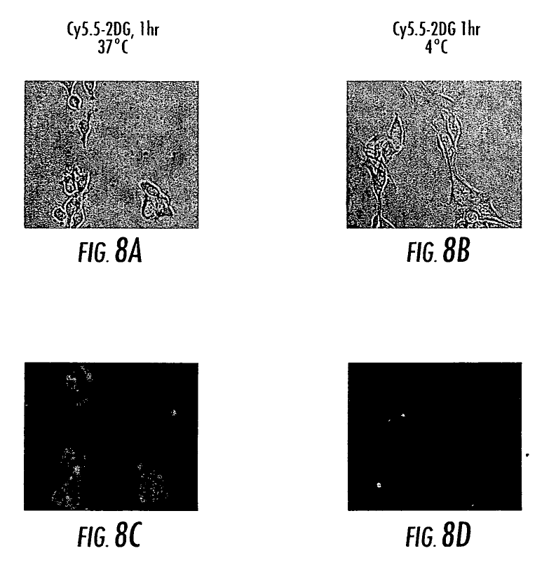

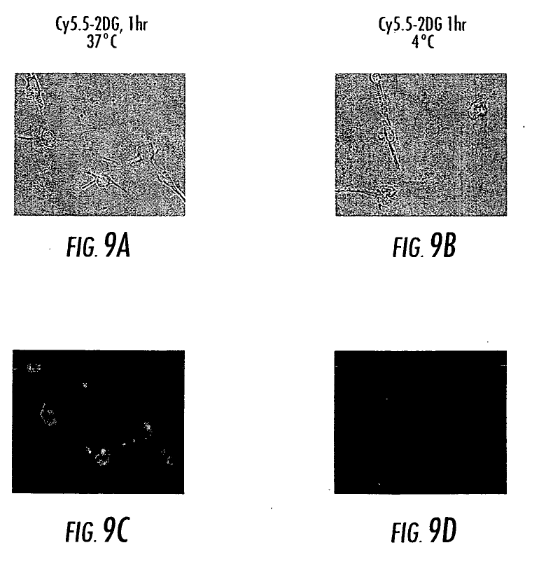

FIGS. 9A-9D illustrate the results of a similar study using MDA-MB-435 human breast carcinoma cells. Incubation at 37° C. (FIGS. 9A, 9C) resulted in uptake of the Cy5.5-2DG compound into the tumor cells, whereas little of the compound was and at 4° C. (FIGS. 9B and 9D).

In vivo studies were done to evaluate performance of the Cy5.5-2DG compound in tumor-bearing subjects. As described in Example 3, mice were inoculated in the right foreleg with 5×10⁶ U87MG cultured glioblastoma cells. Two to three weeks after inoculation, when the tumors reached 0.4-0.6 cm in diameter, the tumor-bearing mice were used for in vivo imaging studies.

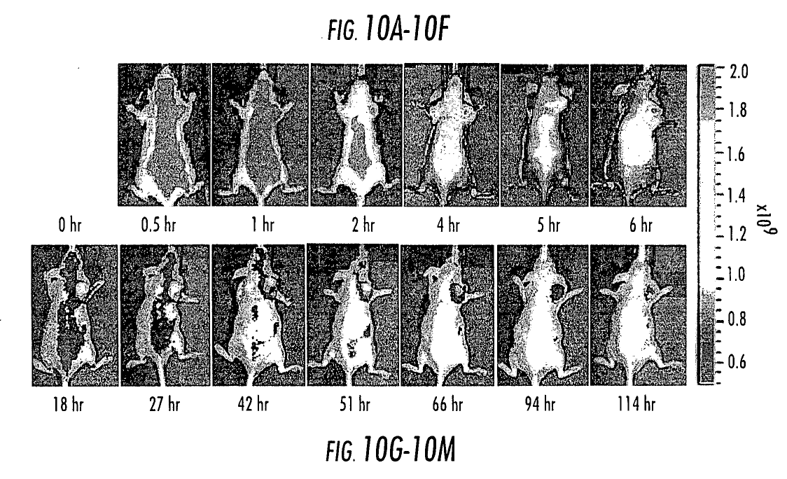

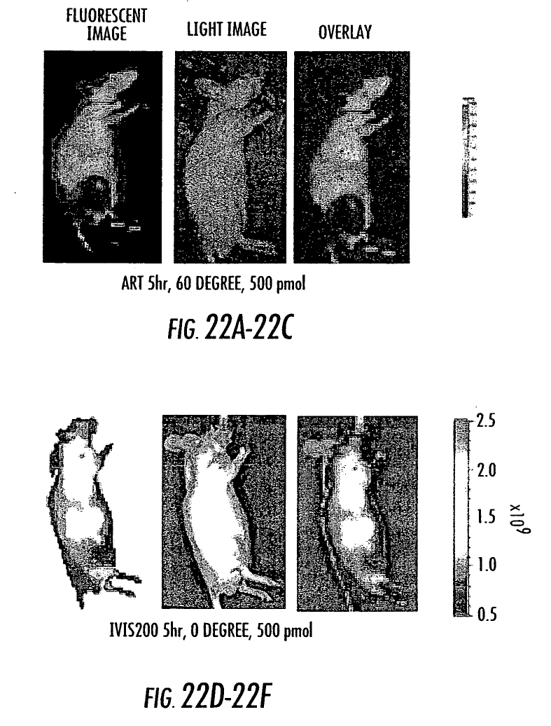

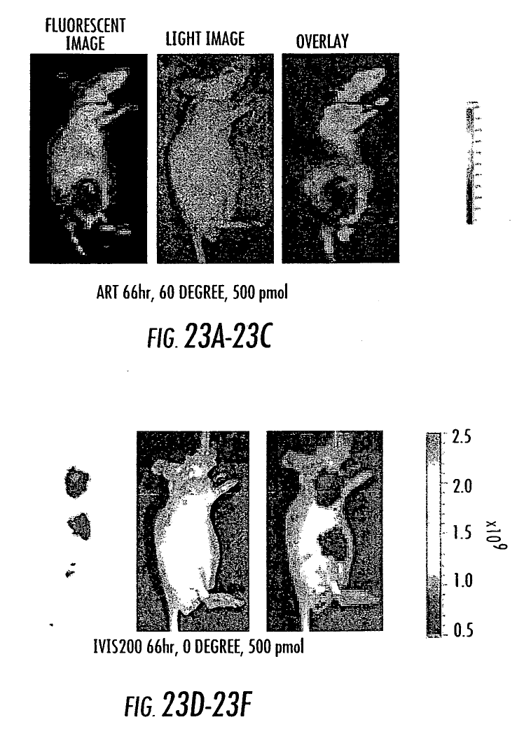

In one study, the mice received 500 pmol of Cy5.5-2DG compound, injected via tail vein. At various times post injection of the probe, an animal was sedated and then imaged using a small animal imaging system available under the trade designation IVIS® (Xenogen, Alameda, Calif.). In other studies described below with respect to FIGS. 22-23, imaging was also done using an imaging system from ART eXplore Optix (ART Inc./GE Healthcare) and compared to imaging using the imaging system IVIS®. FIGS. 10A-10M are images generated with the IVIS® system of sedated, live mice in the prone position as a function of time after administration of 500 pmol of Cy5.5-2DG. FIG. 10A shows the animal 30 minutes post-injection of the compound, and the fluorescence in the body is visible. At 1 hour (FIG. 10B), 2 hours (FIG. 10C), 4 hours (FIG. 10D), and 5 hours (FIG. 10E), the probe progressively clears from the body and begins to localize in the tumor in the right foreleg. In fact, by two hours post injection, accumulation of the compound in the tumor is readily apparent in the image (FIG. 10C). The remaining images in FIG. 10 show the time points corresponding to 6 hours (FIG. 10F), 18 hours (FIG. 10G), 27 hours (FIG. 10H), 42 hours (FIG. 10I), 51 hours (FIG. 10J), 66 hours (FIG. 10K), 94 hours (FIG. 10L), and 114 hours (FIG. 10M) post injection. The presence of the probe in the tumor is apparent in all the images, long after the probe has cleared from healthy tissue.

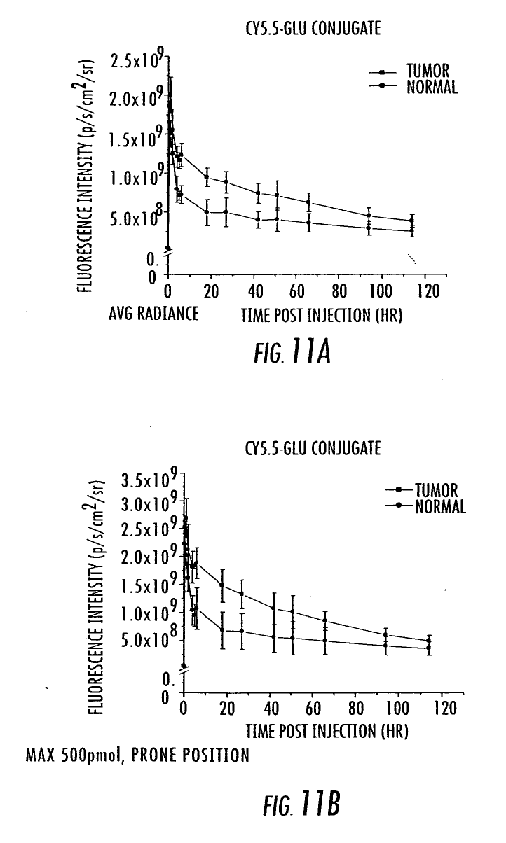

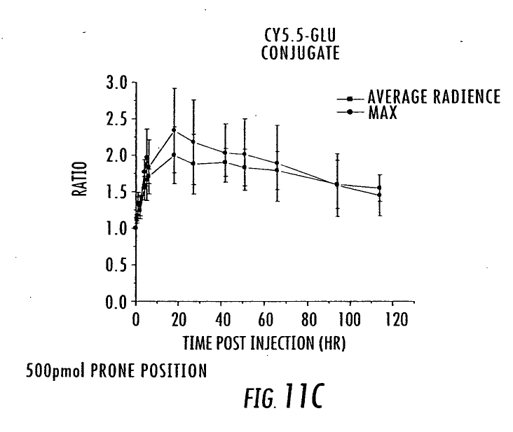

The images at each time in FIGS. 10A-10M were analyzed with suitable software (Living Image®, Xenogen Corp.) to quantify the fluorescence intensity in the tumor tissue and healthy tissue. The results are shown in FIGS. 11A-11B, where fluorescence intensity, in photons/sec/cm²/steradian, is plotted as a function of time post injection of 500 pmol of Cy5.5-2DG, in hours, in normal tissue (circles) and in tumor tissue (squares). FIG. 11A shows the average radiance in the two tissues, and FIG. 11B shows the maximum radiance in the two tissues, at each time point. In both graphs, the fluorescence intensity decreases with time post injection, and the tumor tissue has a consistently higher fluorescence intensity than healthy tissue. FIG. 11C is a graph of the ratio of fluorescence intensity of tumor tissue to the fluorescence intensity of healthy tissue, determined from the data presented in FIGS. 11A-11B, for the average radiance (squares) and for the maximum radiance (circles). Looking at the line corresponding to the average radiance, a ratio of 1.5 is achieved at about 4 hours post-injection, with a ratio of nearly two reached buy about 18 hours post injection. The ratio is relatively constant for up to 66 hours post injection, and then begins to decrease. However, even at 120 hours post injection, a ratio of 1.5 is observed, indicating the presence of the compound in the tumor to a greater extent than in healthy tissue.

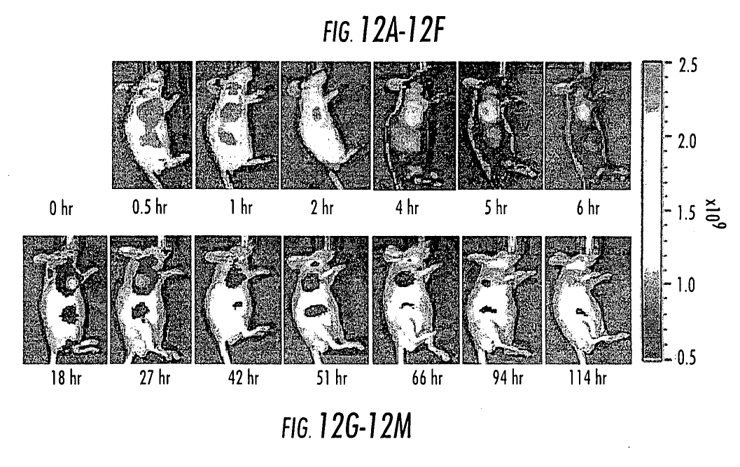

FIGS. 12A-12M show images of the same mouse shown in FIGS. 10A-10M, but positioned laterally. As in the images of FIG. 10, the images were taken as a function of time after administration of 500 pmol of Cy5.5-2DG. Localization of the compound in the tumor is apparent in the image of FIG. 12C, taken two hours post injection of the compound. Fluorescence of the tumor tissue is observed at all time points including the image taken 94 hours post injection (FIG. 12L). The compound appears to have cleared from the tumor by 114 hours post injection, since the tumor is no longer visible in the image of FIG. 12M.

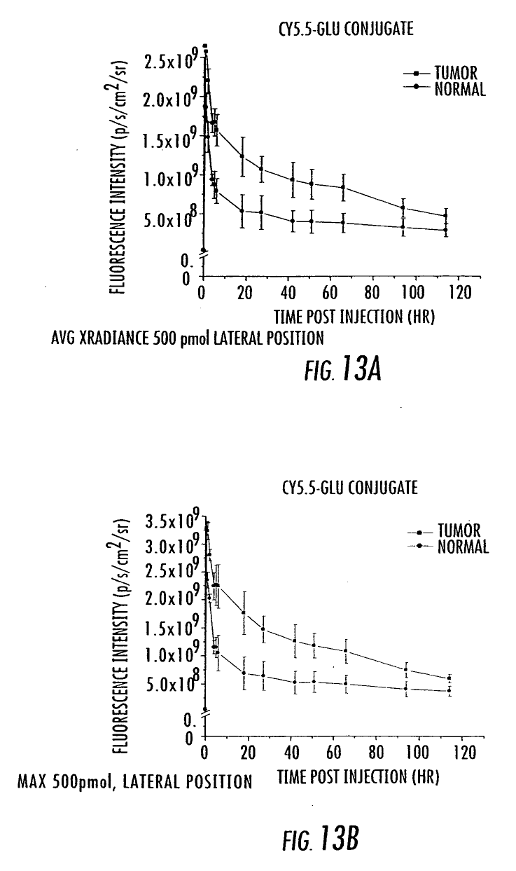

FIGS. 13A-13B are graphs showing the kinetics of the compound in normal tissue (circles) and in tumor tissue (squares), where the fluorescence intensity is quantified as the average radiance fluorescence (FIG. 13A) and the maximum fluorescence (FIG. 13B) for the mice shown in FIG. 12. The fluorescence intensity decreases with time post injection, and the tumor tissue has a consistently higher fluorescence intensity than healthy tissue. FIG. 13C is a plot of the ratio of fluorescence intensity of tumor tissue to the fluorescence intensity of healthy tissue, determined from the data presented in FIGS. 13A-13B, as a function of time post injection of 500 pmol of Cy5.5-2DG, in hours, where the ratio is determined for the measured average radiance fluorescence (squares) and for the maximum fluorescence (circles). The ratio reaches a value of two by about five hours post injection of the probe, and remains around two for up to 100 hours post injection.

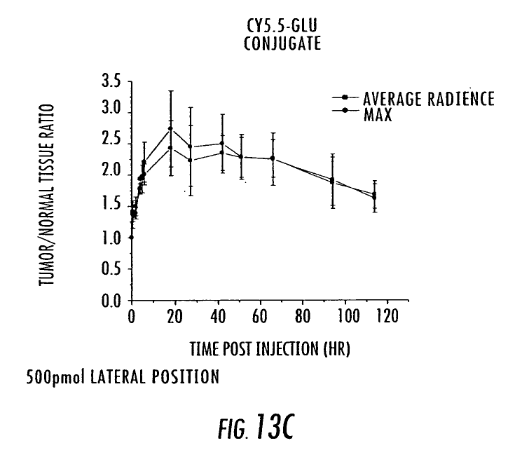

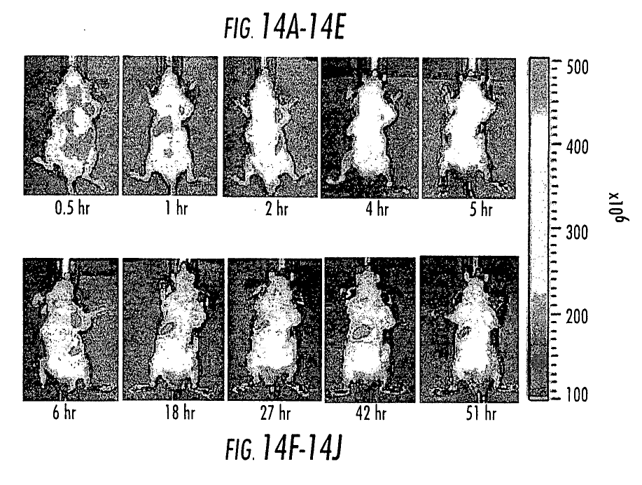

Another study was conducted using mice bearing a human glioblastoma xenograft, where a small dose, 50 pmol, of the Cy5.5-2DG compound was administered. FIGS. 14A-14J are images of a sedated, live mouse in the prone position as a function of time after administration of the fluorochrome probe. With the smaller dose, preferential accumulation of the probe in the tumor is apparent 30 minutes after injection, as seen in the image in FIG. 14A. The tumor continues to fluoresce in the images taken at 1 hours (FIG. 14B), 2 hours (FIG. 14C), 4 hours (FIG. 14D), 5 hours (FIG. 14E), 6 hours (FIG. 14F), 8 hours (FIG. 14G), 27 hours (FIG. 14H), 42 hours (FIG. 14I), and 51 hours (FIG. 14J) after injection. In the images at the later time points (FIGS. 14G, 14H, 14I, and 14J), fluorescence of the spleen on the left side of the animal is also apparent.

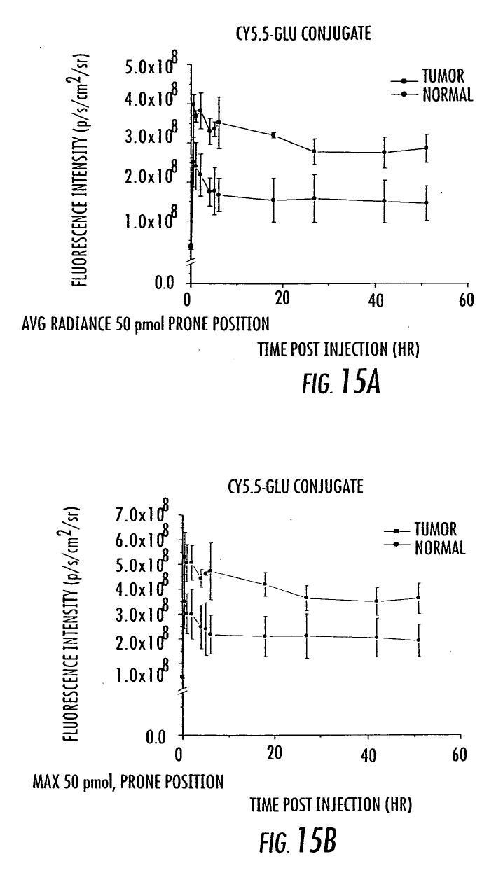

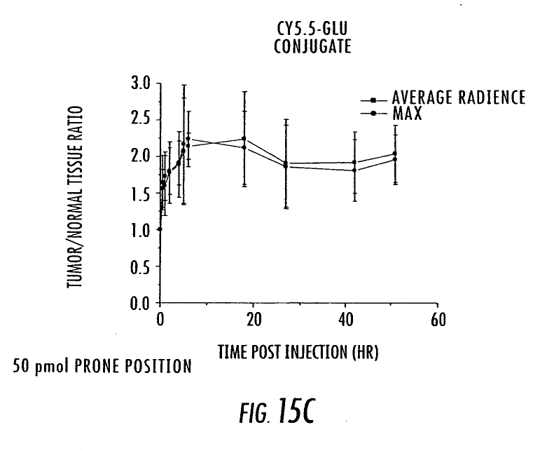

The images in FIGS. 14A-14J were analyzed with suitable software (Living Image®, Xenogen Corp.) to quantify the fluorescence intensity in the tumor tissue and healthy tissue. The results are shown in FIGS. 15A-15B, where fluorescence intensity, in photons/sec/cm²/steradian, is plotted as a function of time post injection of 50 pmol of Cy5.5-2DG, in hours, in normal tissue (circles) and in tumor tissue (squares). FIG. 15A shows the average radiance in the two tissues, and FIG. 15B shows the maximum radiance in the two tissues, at each time point. The fluorescence intensity rapidly reaches a maximum, within 30 minutes of injection of the Cy5.5-2DG probe, and remains elevated for the duration of the study. FIG. 15C is a graph of the ratio of fluorescence intensity of tumor tissue to the fluorescence intensity of healthy tissue, determined from the data presented in FIGS. 15A-15B, for the average radiance fluorescence (squares) and for the maximum fluorescence (circles). A ratio of 1.5 is achieved 30 minutes after injection of the compound. The ratio increases to more than two by about 10 hours after injection and remains elevated for the rest of the time points.

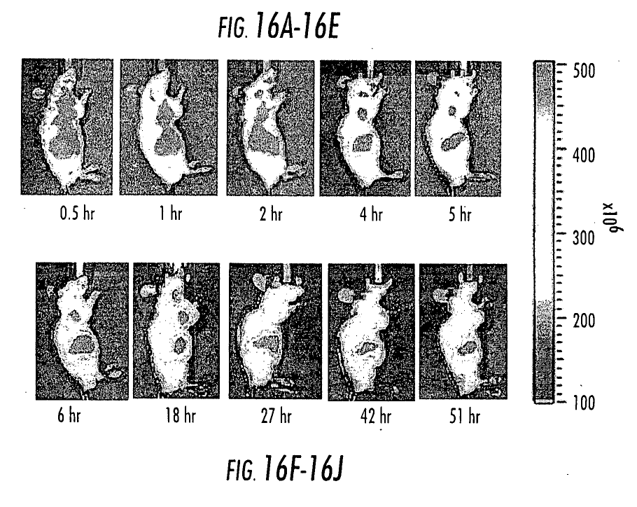

FIGS. 16A-16J correspond to the images of the mouse shown in the prone position in FIGS. 14A-14J, but shown here in the lateral position. Accumulation of the compound in the tumor in the right foreleg is apparent in the image taken 1 hour after injection (FIG. 16B) and remains visible at all subsequent images at the times indicated in FIGS. 16C-16J.

Analysis of the images in FIG. 16 was done, and the graphs are shown in FIGS. 17A-17B, where fluorescence intensity, in photons/sec/cm²/steradian, is plotted as a function of time post injection of 50 pmol of Cy5.5-2DG, in hours, in normal tissue (circles) and in tumor tissue (squares). FIG. 17A shows the average radiance fluorescence intensity, and FIG. 17B shows the maximum. Higher intensity in the tumor tissue relative to the healthy tissue is apparent, with a ratio of about two (FIG. 17C).

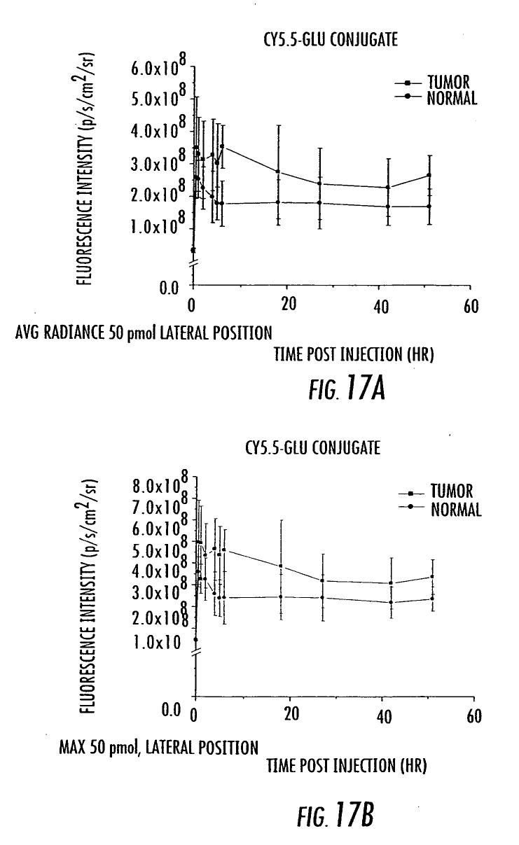

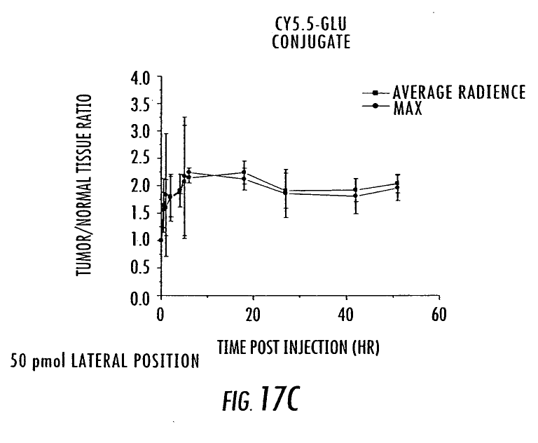

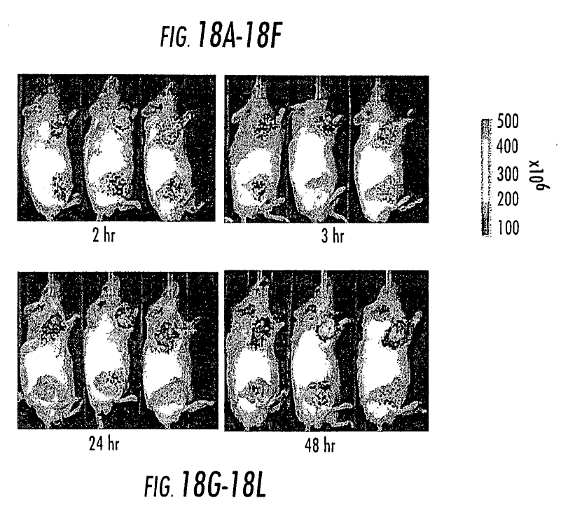

Another study was conducted using mice (n=3) bearing a human melanoma (A375M) xenograft. When the tumor reached 0.4-0.6 mm diameter, the mice received via tail vein injection 500 pmol of Cy5.5-2DG compound. The mice were imaged at 2 hours, 3 hours, 24 hours, and 48 hours post injection, and the results are shown in FIGS. 18A-18L. At 2 hours post-injection (FIGS. 18A-18C) localization of the compound in the tumor is apparent. At 3 hours post-injection (FIGS. 18D-18F), 24 hours post-injection (FIGS. 18G-18I), and 48 hours post-injection (FIGS. 18J-18L) a continued preferential fluorescence of tumor tissue was observed.

In the study described above where a 500 pmol dose of Cy5.5-2DG compound was administered, one mouse was euthanized at 45 hours post injection and the tumor and major tissue and organs were dissected for fluorescence imaging. The image is shown in FIG. 19. The tumor tissue is indicated by the arrow. The image shows that the tumor tissue had the highest fluorescence intensity among the tissues imaged. In the image fields, only the tumor and the stomach exhibited maximum fluorescence.

FIGS. 20A-20F are photomicrographs taken at 10× magnification (FIGS. 20A-20C) and 60× magnification (FIGS. 20D-20F) of tumor tissue 24 hours post-injection of 50 pmol of Cy5.5-2DG. FIGS. 20A and 20D are light images, FIGS. 20B and 20E are fluorescent images, and FIGS. 20C and 20F are overlay images. The images clearly show the boundaries of the tumor, making tumor detection for treatment (such as surgical resection), easier, as will be discussed below.

FIGS. 21A-21F are photomicrographs taken at 10× magnification (FIGS. 21A-21C) and 60× magnification (FIGS. 21D-21F) of tumor tissue 62 hours post-injection of 50 pmol of Cy5.5-2DG. FIGS. 21A and 21D are light images, FIGS. 21B and 21E are fluorescent images, and FIGS. 21C and 21F are overlay images. The boundaries of the tumor remain easily distinguishable 62 hours after administration of the compound.

In the small animal studies described above, a small animal imaging system known under the trade name IVIS® was used. In one study, another imaging system from ART eXplore Optix (ART Inc./GE Healthcare; hereinafter “ART”) was additionally used, and the images from the two systems compared. FIGS. 22A-22F are photomicrographs of sedated, live tumor-bearing mice in the lateral position taken with a small animal imaging system from ART (FIGS. 22A-22C) and with the imaging system IVIS® (FIGS. 22D-22F) system 5 hours after administration of 500 pmol of Cy5.5-2DG. FIGS. 22A-22C correspond to a fluorescent image, a light image, and an overlay image, respectively, using the imaging system from ART. FIGS. 22D-22F correspond to a fluorescent image, a light image, and an overlay image, respectively, using the imaging system known as IVIS®.

FIGS. 23A-23F are photomicrographs taken with the two imaging systems of the mice 66 hours after administration of 500 pmol of Cy5.5-2DG. The mice are sedated and in the lateral position. The photomicrographs were taken with a small animal imaging system from ART (FIGS. 23A-23C) and with the imaging system IVIS® (FIGS. 23D-23F) system 66 hours after administration of 500 pmol of Cy5.5-2DG. FIGS. 23A-23C correspond to a fluorescent image, a light image, and an overlay image, respectively, using the imaging system from ART. FIGS. 23D-23F correspond to a fluorescent image, a light image, and an overlay image, respectively, using the imaging system known as IVIS®.

The images in FIGS. 22-23 illustrate that the Cy5.5-2DG compound is suitable for use with both imaging systems, the tumor in the right foreleg being visible when the animal is imaged with both systems.

The in vitro and in vivo studies described above illustrate utility of the Cy5.5-2DG probe in imaging tumors in vivo in a living subject. The compound preferentially accumulates after administration into the tumor tissue and is retained in the tumor for up to 100 hours, providing a means for imaging as a function of time with a single dose of an imaging agent. Thus, in one aspect, a method for imaging tissue, comprising contacting the tissue with a compound comprised of a cyanine compound attached to a derivative of glucose is provided. Typically, the tissue is a tumor in vivo and is contacted by administering the compound to the subject bearing the tumor. The compound can be administered by any method that enables its delivery to the tumor or cancer site, or to the site suspected of having cancerous tissue. Delivery via circulation in the blood stream, where the compound is injected intravenously is one preferred route; however, other parenteral routes of administration are suitable, such as subcutaneous, intramuscular, and intravascular. The compound can also be administered locally to the cancer site or site of suspected cancer by injection directly into the suspicious tissue.

The amount of compound administered will depend on the subject being treated (weight, age, condition), the severity of the cancer, the location of the cancer, the rate of administration, and other factors discerned by the medical provider.

It will be appreciated that the compound can be formulated with pharmaceutically-acceptable excipients suitable for the selected mode of administration. For intravenous administration, the compound can be formulated as a solution or suspension of the compound in sterile aqueous solutions of, for example, saline, dextrose, or propylene glycol. Methods of preparing pharmaceutical formulations with a specific amount of the imaging probe are apparent to those skilled in the art, and examples are provided in recognized texts, such as Remington's Pharmaceutical Sciences.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure and protected by the following claims.

We claim:

1. A compound, comprising
a cyanine/glucose compound, wherein the cyanine/glucose compound includes a cyanine compound that fluoresces in the near infrared region attached to a glucose compound.

2. The compound of claim 1, wherein the cyanine/glucose compound is a compound having structure I: 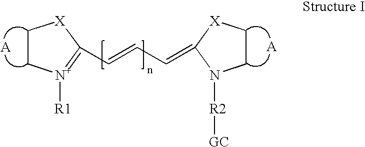
wherein X is selected from one of the following: O, NR, C(CH₃)₂, and S; wherein R, R1, and R2 are each independently selected from one of the following: an alky group, an aryl group, a substituted alkyl, and a substituted aryl; wherein A is selected from a cyclic multi-ring and a fused multi-ring; wherein n is 1, 2, or 3; and wherein GC is the glucose compound.

3. The compound of claim 1, wherein the cyanine/glucose compound is a compound having structure II: 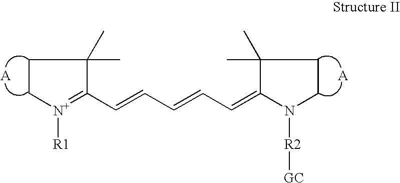
wherein R1 and R2 are each independently selected from one of the following: an alky group, an aryl group, a substituted alkyl, and a substituted aryl; wherein A is selected from a cyclic multi-ring and a fused multi-ring; and wherein GC is the glucose compound.

4. The compound of claim 1, wherein the cyanine/glucose compound is a compound having structure III: 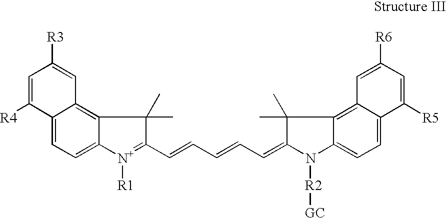
wherein R1 and R2 are each independently selected from one of the following: an alky group, an aryl group, a substituted alkyl, and a substituted aryl; wherein R3, R4, R5, and R6 are each independently selected from a sulfonic acid group and sulfonate group; and wherein GC is the glucose compound.

5. The compound of claim 1, wherein the cyanine/glucose compound is a compound having structure IV: 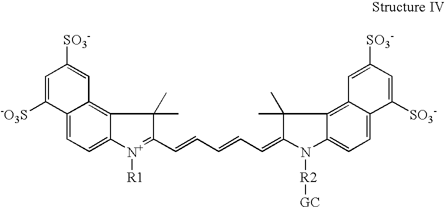
wherein R1 and R2 are each independently selected from one of the following: an alky group, an aryl group, a substituted alkyl, and a substituted aryl; and wherein GC is the glucose compound.

6. The compound of claim 1, wherein the cyanine/glucose compound is a compound having structure V: 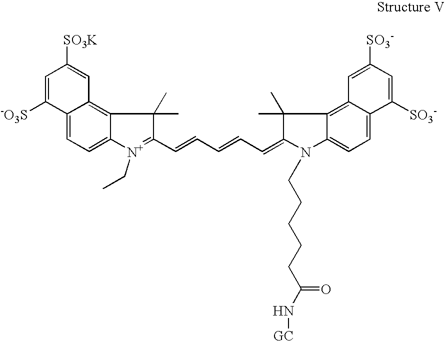
wherein GC is the glucose compound.

7. The compound of claim 1, wherein the cyanine compound fluoresces from about 650 to 900 nm.

8. The compound of claims 1, wherein the glucose compound is selected from one of the following: glucose and derivatives thereof, D-glucose and derivatives thereof, glucosamine and derivatives thereof.

9. The compound of claim 1 wherein the cyanine compound is attached to the glucose compound by an amide linkage.

10. A method for imaging tissue, comprising contacting a tissue with a compound comprising a compound of claims 1.

11. The method of claim 10, wherein the contacting comprises contacting precancerous cells, cancer, or tumor tissue in vitro with the compound.

12. The method of claim 10, wherein the contacting comprises contacting precancerous cells, cancer, or tumor tissue tissue in vivo with the compound.

13. The method of claim 10, wherein the contacting is achieved by intravenous administration of the compound to a subject.

14. The method of claim 10, wherein the contacting is achieved by local infusion of the compound to a subject.

15. The method of claim 10, further comprising imaging the tissue.

16. The method of claim 10, further comprising: diagnosing the presence of one or more of precancerous cells, cancerous cells, and tumor cells in the tissue.

17. The method of claim 10, further comprising: monitoring the progress of one or more of precancerous cells, cancerous cells, and tumor cells in the tissue before and after administration of a drug.

18. The method of claim 10, further comprising: monitoring the progress of one or more of precancerous cells, cancerous cells, and tumor cells in the tissue before and after administration of chemotherapy.

19. A fluorescent probe prepared according to a process comprising: reacting a cyanine compound having a moiety with a glucose compound having a moiety, wherein the moiety of the cyanine compound is capable of reacting with the moiety of the glucose compound to form a cyanine/glucose compound.

20. A fluorescent probe prepared according to a process comprising: reacting a cyanine compound having a moiety capable of reacting with an amine with a glucose compound having an amine moiety.

21. The method of claim 18, wherein the cyanine compound has an N-hydroxysuccinimide ester moiety and the glucose is glucosamine.
